(19) United States
(12) Patent Application Publication
(10) Pub. No.: US 2026/0193720 A1
(43) Pub. Date: Jul. 9, 2026

(54) METHODS OF DETERMINING VARIABILITY IN MOLECULAR ALTERATIONS

(71) Applicant: Ellison Institute, LLC, Los Angeles, CA (US)

(72) Inventors: Naim Matasci, Los Angeles, CA (US); David B. Agus, Los Angeles, CA (US)

(21) Appl. No.: 19/559,043

(22) Filed: Mar. 6, 2026

Related U.S. Application Data
(63) Continuation of application No. PCT/US2024/045593, filed on Sep. 6, 2024.
(60) Provisional application No. 63/581,553, filed on Sep. 8, 2023.

(51) Int. Cl. C12Q 1/6886; C12Q 1/6809; C12Q 1/6869

(52) U.S. Cl. CPC C12Q 1/6886; C12Q 1/6809; C12Q 1/6869; C12Q 2600/154; C12Q 2600/156

(57) ABSTRACT

Described and provided herein are methods and systems useful for the analysis of polynucleotides and/or detection/quantification of cancer risk identifiers for cancer screening. For example, in certain instances, the methods and systems described are useful for determining changes in the rates of genetic change (e.g., mutation accumulation as a function of time) and/or epigenetic changes (e.g., methylation status change as a function of time), which can be used to profile the risk that an individual has cancer or is likely to develop cancer.

### CROSS REFERENCE

This application is continuation of International Application No. PCT/US2024/045593, filed Sep. 6, 2024, which claims the benefit of U.S. Provisional Ser. No. 63/581,553 filed on Sep. 8, 2023, which is incorporated by reference herein in its entirety.

### BACKGROUND

Cancer prevention is marked by several formidable challenges, namely poor compliance and a ‘one size fits all’ approach. There are national standards for cancer prevention and screening, but they rarely reflect the risk of the individual. A yearly assessment of cancer risk is critical to making personalized prevention and screening program.

### SUMMARY

Current technologies and studies generally fail to assess the dynamic aspect of the accumulation genomic and epigenomic changes (e.g., changes in rates and velocities of mutation accumulation) and whether such rates are responsive to environmental and physiological changes. Throughout human life, healthy cells accumulate somatic mutations due to intrinsic and extrinsic forces, a process that varies between both individuals and tissues. Studies have shown that mutations involved in the regulation of cell processes and associated with the development of cancer as precursors, sometimes years before the appearance of the disease. The human body is generally primed to prevent those multiple mutations from leading to cancer through critical cellular regulation routes. However, when the body is under acute or chronic stress, those safeguarding process may become less dependable and the affected tissues may be more prone to develop cancer. Decades of research have revealed mechanisms of DNA repair and replication in mutagenesis, yet surprisingly little is known about the rate of accumulation of somatic mutations caused by normal cellular processes over an individual's lifetime and its fluctuations in response to intrinsic or extrinsic factors.

Population-scale data have revealed that on average, ~1.3 somatic exonic mutations are acquired per hematopoietic stem cell per decade. As hematopoietic cells travel to different tissues of the body, affecting homeostasis among a number of different organ systems cells. Recent studies have demonstrated the potential for changes in such homeostasis to impact risk for cardiovascular disease, diabetes, cancer, and other diseases and/or disorders. Initial efforts sequencing of peripheral blood DNA in a cohort of 12,380 Swedish patients was among the first to identify a large fraction of clonal hematopoiesis (CH) carriers that did not have known disease driving mutations. Similar results were found in ~11,262 Icelandic patients using short-read sequencing. Unidimensional studies a study using long-read sequencing (LRS) of whole blood and heart tissue from ~3,622 patients have also provided preliminary evidence that application of LRS can be used to probe the effects sequence variation on disease phenotype.

However, such studies fail to assess the dynamic aspect of mutation accumulation and whether its rate is responsive to environmental changes. These studies also emphasize the need to better understand the process of accumulation of somatic mutations and their effect on general health. The lack of serial longitudinal samples have limited study designs and methodologies used to establish these associations as genome sequences and disease (e.g., cancer) risk vary between ancestry groups. As studies detail new genotypic and phenotypic associations, researchers and clinicians are confronted with the question of what preventative and therapeutic interventions could be taken to mitigate disease risk.

Provided and described herein methods for measuring and characterizing the mutation accumulation process over time (e.g., velocities of mutation accumulation), and in certain instances, such methods serve as a surrogate for the state of the system of an individual, and to inform clinical course trajectories through this monitoring, and ultimately to evaluate useful interventions for changing their outcome.

In some embodiments, provided and described herein are methods of screening for cancer in an individual, the method comprising: (a) sequencing polynucleotides from the genome of the individual at a first timepoint and generating: (i) sequencing reads associated with the first timepoint, and (ii) a first rate of genomic mutation accumulation using (1) the sequencing reads associated with the first timepoint and (2) a reference sequence; and (b) sequencing polynucleotides from the genome of the individual at a second timepoint and generating: (i) sequencing reads associated with the second timepoint, and (ii) a second rate of genomic mutation accumulation using (1) the sequencing reads associated with the second timepoint and (2) the sequencing reads associated with the first timepoint.
In some embodiments, provided and described herein are methods of quantifying somatic mutations in a genome of an individual, the method comprising: (a) sequencing polynucleotides from the genome of the individual at a first timepoint and generating: (i) sequencing reads associated with the first timepoint, and (ii) a first rate of mutation accumulation using (1) the sequencing reads associated with the first timepoint and (2) a reference sequence; and (b) sequencing polynucleotides from the genome of the individual at a second timepoint and generating: (i) sequencing reads associated with the second timepoint, and (ii) a second rate of mutation accumulation using (1) the sequencing reads associated with the second timepoint and (2) the sequencing reads associated with the first timepoint.

In some embodiments, further described and provided are methods of processing polynucleotides from the genome of an individual useful for quantifying somatic SNV accumulation, the method comprising: (a) longitudinally collecting three or more blood and/or plasma samples from the individual at three or more timepoints; (b) sequencing polynucleotides from the genome of the individual in the three or more blood and/or plasma samples; (c) generating a first rate of somatic SNV accumulation and a second rate of mutation accumulation, wherein: (i) the first rate of somatic mutation accumulation comprises the number of somatic SNV mutations present in sequencing reads associated with a second timepoint relative to a first timepoint, and (ii) the second rate of somatic mutation accumulation comprises the number of somatic SNV mutations present in sequencing reads associated with a third timepoint relative to the sequencing reads associated with the second timepoint. In certain instances, such methods are useful for quantifying somatic SNV accumulation indicative of increased cancer risk.

In some embodiments, provided and described herein are methods of detecting cancer, comprising: administering a cancer type-specific test to an individual having an increased quantity or rate of mutation accumulation, wherein the increased quantity or rate of mutation accumulation is or has been determined by: a rate of mutation accumulation using (1) sequencing reads from the genome of the individual associated with a first timepoint and (2) sequencing reads from the genome of the individual associated with a second timepoint, wherein the quantity or rate of mutation accumulation is greater than a reference rate.

In some embodiments, provided are methods of detecting cancer, comprising: administering a cancer type-specific test to an individual having an increased quantity or rate of methylation status change, wherein the increased quantity or rate of methylation status change is or has been determined by: a rate of methylation status change using (1) sequencing reads from the genome of the individual associated with a first timepoint and (2) sequencing reads from the genome of the individual associated with a second timepoint, wherein the quantity or rate of methylation status change is greater than a reference rate.

In some embodiments, provided are methods of detecting cancer, comprising: administering a cancer type-specific test to an individual having an increased quantity or rate of mutation accumulation and methylation status change, wherein the increased quantity or rate of mutation accumulation and methylation status change is or has been determined by: a rate of mutation accumulation using (1) sequencing reads from the genome of the individual associated with a first timepoint and (2) sequencing reads from the genome of the individual associated with a second timepoint, wherein the quantity or rate of mutation accumulation is greater than a reference rate; and a rate of methylation status change using (1) sequencing reads from the genome of the individual associated with a first timepoint and (2) sequencing reads from the genome of the individual associated with a second timepoint, wherein the quantity or rate of methylation status change is greater than a reference rate.

### BRIEF DESCRIPTION OF THE DRAWINGS

The novel features of the disclosure are set forth with particularity in the detailed description and appended claims. A better understanding of the features and advantages of the present invention will be obtained by reference to the following detailed description that sets forth illustrative embodiments, in which the principles of the invention are utilized, and the accompanying drawings of which:

FIG. 1A shows recovery of single nucleotide variants observed in a pure sample of BT-474 DNA at 1%, 5% and 10% DNA spike-n concentration, using long-read sequencing (LRS).

FIGS. 1B, 1C, 1D, 1E, and 1F show identification of characteristic SBS mutational signatures of BT-474 at different DNA spike-in concentrations and retrieval of BT-474 characteristic signatures (SBS2, SBS13) and universal SBS (SBS1, SBS5) as a function of BT-474 spike-in concentration.

FIGS. 2A and 2B show sSNVs variant allele frequency density plot before filtering (2A top) and after filtering (2A bottom). Before filtering ~1 million putative somatic SNVs per sample. After filtering ~6,000 high-confidence somatic SNVs per sample.

FIG. 3 shows TensorSignatures of subjects' high-confidence sSNVs compared to HG002 false positives sSNVs.

FIG. 4 shows COSMIC SBS signatures distribution across subject samples (“post-filtering”) and false positive calls from HG002 sequencing.

FIG. 5 shows corrected counts of high-confidence sSNVs increase linearly with subject's age.

FIGS. 6A, 6B, 6C, and 6D show short term changes in mutational signatures.

FIG. 7 shows the single nucleotide variant filtration scheme.

FIGS. 8A and 8B show representations of the pilot study design and overview.

FIG. 9 shows variant calling pipeline schema.

FIG. 10 shows SNV filtration isolates a distinct allele frequency distribution for somatic variants.

FIGS. 11A and 11B shows for putative somatic SNV counts over time for control and treatment (cancer positive) cohorts.

FIGS. 12A and 12B shows for putative somatic SNV counts and mutation rates time for control and treatment (cancer positive) cohorts.

### DETAILED DESCRIPTION

Described and provided herein are methods and systems for the analysis of polynucleotides and/or detection/quantification of cancer risk identifiers. For example, in certain instances, the methods and systems described are useful for determining changes in the rates of genetic change (e.g., mutation accumulation as a function of time) and/or epigenetic changes (e.g., methylation status change as a function of time), which can be used to profile the risk that an individual has cancer or is likely to develop cancer.

In some embodiments, provided and described herein are methods of screening for cancer in an individual, the method comprising:
(a) sequencing polynucleotides from the genome of the individual at a first timepoint and generating:
(i) sequencing reads associated with the first timepoint, and
(ii) a first rate of genomic mutation accumulation using (1) the sequencing reads associated with the first timepoint and (2) a reference sequence; and
(b) sequencing polynucleotides from the genome of the individual at a second timepoint and generating:
(i) sequencing reads associated with the second timepoint, and
(ii) a second rate of genomic mutation accumulation using (1) the sequencing reads associated with the second timepoint and (2) the sequencing reads associated with the first timepoint.

In some embodiments, provided and described herein are methods of quantifying somatic mutations in a genome of an individual, the method comprising:
(a) sequencing polynucleotides from the genome of the individual at a first timepoint and generating:
(i) sequencing reads associated with the first timepoint, and
(ii) a first rate of mutation accumulation using (1) the sequencing reads associated with the first timepoint and (2) a reference sequence; and
(b) sequencing polynucleotides from the genome of the individual at a second timepoint and generating:
(i) sequencing reads associated with the second timepoint, and
(ii) a second rate of mutation accumulation using (1) the sequencing reads associated with the second timepoint and (2) the sequencing reads associated with the first timepoint.

In some embodiments, further described and provided are methods of processing polynucleotides from the genome of an individual useful for quantifying somatic SNV accumulation, the method comprising: (a) longitudinally collecting three or more blood and/or plasma samples from the individual at three or more timepoints; (b) sequencing polynucleotides from the genome of the individual in the three or more blood and/or plasma samples; (c) generating a first rate of somatic SNV accumulation and a second rate of mutation accumulation, wherein: (i) the first rate of somatic mutation accumulation comprises the number of somatic SNV mutations present in sequencing reads associated with a second timepoint relative to a first timepoint, and (ii) the second rate of somatic mutation accumulation comprises the number of somatic SNV mutations present in sequencing reads associated with a third timepoint relative to the sequencing reads associated with the second timepoint. In certain instances, such methods are useful for quantifying somatic SNV accumulation indicative of increased cancer risk.

In some embodiments, described and provided are methods of detecting cancer in an individual, the method comprising: administering a cancer type-specific test to an individual having an increased rate of somatic mutation accumulation, wherein the increased rate of somatic mutation accumulation is or has been determined by:
a second rate of somatic mutation accumulation being greater than a first rate of somatic mutation accumulation, wherein:
the first rate of somatic mutation accumulation comprises the number of somatic mutations present in sequencing reads associated with a first timepoint relative to a reference sequence,
and the second rate of somatic mutation accumulation comprises the number of somatic mutations present in sequencing reads associated with a second timepoint relative to the sequencing reads associated with the first timepoint.

In certain instances, the methods described herein are useful for identifying an individual as having an increased risk of cancer. In certain embodiments, the methods further comprise, identifying the individual as having an increased risk of cancer if the second rate of mutation accumulation is greater than the first rate of mutation accumulation.

Generally, single nucleotide variation (SNV) calling is used to assess mutation accumulation and generate rates of mutation accumulation. In certain instances SNV calling utilizes an aggressive set of filters to call high quality somatic SNVs. In certain embodiments, the methods comprise: generating the first rate of mutation accumulation comprises generating the number of mutations present in the sequencing reads associated with the first timepoint relative to the reference sequence; and generating the second rate of mutation accumulation comprises generating the number of mutations present in the sequencing reads associated with the second timepoint relative to the sequencing reads associated with the first timepoint. In certain embodiments, the genomic mutation accumulation comprises the accumulation of somatic single nucleotide variants (SNVs) present in the sequencing reads. In certain embodiments, the somatic SNVs are high-confidence somatic SNVs. In certain embodiments, the high-confidence somatic SNVs are obtained by removing SNVs associated with sequencing artifacts detected in control sequencing reads from a control genome, removing putative germline SNVs, removing SNVs having a frequency equal to or greater than about 30% to 40%, removing SNVs mapping to known single nucleotide polymorphisms having an allele frequency greater than 0% (e.g., equal to or greater than 0.01%), or a combination thereof. In certain embodiments, the high-confidence somatic SNVs are obtained by removing SNVs associated with sequencing artifacts detected in control sequencing reads from a control genome, removing putative germline SNVs, removing SNVs having a frequency equal to or greater than about 40%, and removing SNVs mapping to known single nucleotide polymorphisms having an allele frequency greater than about 0% (e.g., equal to or greater than 0.01%).

In certain embodiments, the substantially all germline SNVs or all germline SNVs are known for the control genome. Exemplary control genomes include HG001 and HG002. In certain embodiments, the control genome is sequenced in parallel with the (a) and/or (b). For example, in certain embodiments, the methods further comprises: sequencing polynucleotides from a control genome at the first timepoint and/or the second timepoint, and generating control sequencing reads associated with the first timepoint and/or second timepoint, wherein all or substantially all of the sequence of the control genome is known, including all or substantially all germline SNVs. In certain embodiments, the methods further comprise:
generating a set of variants associated with sequencing artifacts by:
(i) filtering out the known germline SNVs from the control sequencing reads associated with the first timepoint and/or second timepoint; and
(ii) using the sequence of the control genome to identify variants associated with sequencing artifacts in the control sequencing reads associated with the first timepoint and/or second timepoint.

In certain embodiments, generating the first rate of somatic SNV accumulation and/or the second rate of somatic SNV accumulation comprises: (i) filtering out the putative germline SNVs from the sequencing reads associated with the first timepoint and/or second timepoint; (ii) removing variants present in the sequencing reads associated with the first timepoint and/or second timepoint that map to the variants associated with sequencing artifacts in the control sequencing reads associated with the first timepoint and/or second timepoint; (iii) removing SNVs having a frequency equal to or greater than about 40%; and/or (iv) removing SNVs mapping to known single nucleotide polymorphisms having an allele frequency greater than about 0% (e.g., equal to or greater than 0.01%). In certain embodiments, the sequencing comprises long-read sequencing.

Epigenetic changes can be measured in combination with genomic changes. In some embodiments, the methods further comprise: generating a first rate of epigenetic changes associated with the first timepoint and a second rate of epigenetic changes associated with the second timepoint. In certain embodiments, the epigenetic changes comprises changes in genome methylation.

In some embodiments, the methods further comprises administering or performing a cancer type-specific test if the rate of mutation accumulation is greater than to a reference rate.

In some embodiments, provided and described herein are methods of quantifying identifiers of cancer risk in an individual, comprising: (a) sequencing polynucleotides from the genome of the individual at a first timepoint and generating sequencing reads associated with the first timepoint; and (b) sequencing polynucleotides from the genome of the individual at a second timepoint and generating sequencing reads associated with the second timepoint; and (c) generating a rate of mutation accumulation using (1) the sequencing reads associated with the second timepoint and (2) the sequencing reads associated with the first timepoint, and using the rate of mutation accumulation as an identifier of cancer risk. In certain embodiments, the method further comprises after (c): identifying the individual as having an increased risk of cancer if the rate of mutation accumulation is greater than a reference rate. In certain embodiments, the method further comprises: (d) administering a cancer type-specific test if the rate of mutation accumulation is greater than to a reference rate.

In certain embodiments, the method further comprises (d) generating a rate of methylation status change using (1) the sequencing reads associated with the second timepoint and (2) the sequencing reads associated with the first timepoint, and using the rate of methylation status change as an identifier of cancer risk. In certain embodiments, the method further comprises administering a cancer type-specific test if the rate of mutation accumulation or methylation status change is greater than to a reference rate. In certain embodiments, the sequencing comprises sequencing specific loci, and wherein the specific loci comprise (i) a region having an increased frequency of mutations, (ii) an increased rate of local mutation accumulation (e.g., compared to other regions of the genome), or (iii). an increased rate of methylation status change.

In some embodiments, provided are methods of screening for cancer in an individual, comprising: (a) sequencing polynucleotides from the genome of the individual at a first timepoint and generating: (i) sequencing reads associated with the first timepoint, and (ii) a first rate of mutation accumulation using (1) the sequencing reads associated with the first timepoint and (2) a reference sequence; and (b) sequencing polynucleotides from the genome of the individual at a second timepoint and generating: (i) sequencing reads associated with the second timepoint, and (ii) a second rate of mutation accumulation using (1) the sequencing reads associated with the second timepoint and (2) the sequencing reads associated with the first timepoint.

In certain embodiments, the method further comprises, after (b), identifying the individual as having an increased risk of cancer if the second rate of mutation accumulation is greater than the first rate of mutation accumulation. In certain embodiments, the method further comprises: (c) administering a cancer type-specific test if the second rate of mutation accumulation is greater than the first rate of mutation accumulation.

In certain embodiments, the method further comprises in (a): generating (iii) a first rate of methylation status change using (1) the sequencing reads associated with the first timepoint and (2) a reference sequence; and in (b): generating (iii) a second rate of methylation status change using (1) the sequencing reads associated with the first timepoint and (2) the sequencing reads associated with the first timepoint.

In certain embodiments, the sequencing comprises sequencing specific loci, and wherein the specific loci comprise (i) a region having an increased frequency of mutations, (ii) an increased rate of local mutation accumulation (e.g., compared to other regions of the genome), or (iii). an increased rate of methylation status change.

In some embodiments, provided are methods of quantifying identifiers of cancer risk in an individual, comprising: (a) generating a rate of mutation accumulation using (1) sequencing reads from the individual associated with a first timepoint and (2) sequencing reads from the individual associated with a second timepoint; and (b) administering a cancer type-specific test if the rate of mutation accumulation is greater than a reference rate of mutation accumulation.

In some embodiments, provided are methods of quantifying identifiers of cancer risk in an individual, comprising: (a) generating a rate of methylation status change using (1) sequencing reads from the individual associated with a first timepoint and (2) sequencing reads from the individual associated with a second timepoint; and (b) administering a cancer type-specific test if the rate of methylation status change is greater than a reference rate of methylation status change.

In some embodiments, provided are methods of quantifying identifiers of cancer risk in an individual, comprising: (a) generating a rate of mutation accumulation using (1) sequencing reads from the individual associated with a first timepoint and (2) sequencing reads from the individual associated with a second timepoint (b) generating a rate of methylation status change using (1) sequencing reads from the individual associated with the first timepoint and (2) sequencing reads from the individual associated with the second timepoint; and (c) administering a cancer type-specific test if the rate of mutation accumulation is greater than a reference rate of mutation accumulation and/or if the rate of methylation status change is greater than a reference rate of methylation status change.

In certain embodiments, the sequencing reads comprise sequence data for non-cancer risk-associated genes. In certain embodiments, the sequencing reads comprise sequence data for specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing reads comprise sequence data for intronic regions. In certain embodiments, the rate of mutation accumulation comprises rate data for a total number of mutations as a function of time, or wherein the rate of methylation status change comprises rate data for a total number of methylation status changes as a function of time.

In some embodiments, provided and described herein are methods of processing polynucleotides from the genome of an individual useful for quantifying mutation accumulation in the genome, comprising: (a) sequencing the polynucleotides from the genome of the individual at a first timepoint, and generating sequencing reads associated with the first timepoint; (b) sequencing the polynucleotides from the genome of the individual at a second timepoint, and generating sequencing reads associated with the second timepoint; and (c) quantifying the mutation accumulation in the genome at the first timepoint and the second timepoint and generating a rate of mutation accumulation using (1) the sequencing reads associated with the second timepoint and (2) the sequencing reads associated with the first timepoint.

In some embodiments, provided and described herein are methods of processing polynucleotides from the genome of an individual useful for quantifying methylation status change in the genome, comprising: (a) sequencing the polynucleotides from the genome of the individual at a first timepoint, and generating sequencing reads associated with the first timepoint; (b) sequencing the polynucleotides from the genome of the individual at a second timepoint, and generating sequencing reads associated with the second timepoint; and (c) quantifying the methylation status change in the genome at the first timepoint and the second timepoint and generating a rate of methylation status change using (1) the sequencing reads associated with the second timepoint and (2) the sequencing reads associated with the first timepoint.

In some embodiments, provided and described herein are methods of processing polynucleotides from the genome of an individual useful for quantifying mutation accumulation and methylation status change in the genome, comprising: (a) sequencing polynucleotides from the genome of the individual at a first timepoint, and generating sequencing reads associated with the first timepoint; (b) sequencing polynucleotides from the genome of the individual at a second timepoint, and generating sequencing reads associated with the second timepoint; (c) quantifying the mutation accumulation in the genome at the first timepoint and the second timepoint and generating a rate of mutation accumulation using (1) the sequencing reads associated with the second timepoint and (2) the sequencing reads associated with the first timepoint; and (d) quantifying the methylation status change in the genome at the first timepoint and the second timepoint and generating a rate of methylation status change using (1) the sequencing reads associated with the second timepoint and (2) the sequencing reads associated with the first timepoint.

In certain embodiments, the sequencing reads comprise sequence data for non-cancer risk-associated genes. In certain embodiments, the sequencing reads comprise sequence data for specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing reads comprise sequence data for intronic regions. In certain embodiments, the rate of mutation accumulation comprises rate data for a total number of mutations as a function of time, or wherein the rate of methylation status change comprises rate data for a total number of methylation status changes as a function of time. In certain embodiments, the sequencing comprises sequencing non-cancer risk-associated genes. In certain embodiments, the sequencing comprises sequencing specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing comprises sequencing intronic regions. In certain embodiments, the sequencing comprises long read sequencing. In certain embodiments, the polynucleotides comprise genomic DNA or amplification products thereof.

In some embodiments, provided and described herein are methods of processing nucleic acid molecules from the genome of an individual (e.g., including amplification products thereof) useful for quantifying mutation accumulation and/or methylation status change in the genome, the method comprising: (a) sequencing polynucleotides from the genome of the individual at a first timepoint, and generating sequencing reads associated with the first timepoint; (b) sequencing polynucleotides from the genome of the individual at a first timepoint, and generating sequencing reads associated with the second timepoint; and identifying/analyzing mutation accumulation or methylation status change in the genome at the first timepoint and the second timepoint, and generating a quantity or rate of mutation accumulation and/or methylation status change using (1) the sequencing reads associated with the second timepoint and (2) the sequencing reads associated with the first timepoint.

In some embodiments, provided and described herein are methods of processing nucleic acid molecules from the genome of an individual (e.g., including amplification products thereof) useful for quantifying mutation accumulation and/or methylation status change in the genome, the method comprising: (a) sequencing polynucleotides from the genome of the individual at a first timepoint, and generating: sequencing reads associated with the first timepoint and a first quantity or rate of mutation accumulation and/or methylation status change using (1) the sequencing reads associated with the first timepoint and (2) reference sequencing reads; (b) sequencing polynucleotides from the genome of the individual at a second timepoint, and generating: sequencing reads associated with the second timepoint and a second quantity or rate of mutation accumulation and/or methylation status change using (1) the sequencing reads associated with the second timepoint and (2) the sequencing reads associated with the first timepoint.

In some embodiments, provided are methods of quantifying identifiers of cancer risk in an individual, comprising: (a) generating a rate of mutation accumulation using (1) sequencing reads from the individual associated with a first timepoint and (2) sequencing reads from the individual associated with a second timepoint; and (b) administering a cancer type-specific test if the rate of mutation accumulation is greater than a reference rate of mutation accumulation.

In some embodiments, provided are methods of quantifying identifiers of cancer risk in an individual, comprising: (a) generating a rate of methylation status change using (1) sequencing reads from the individual associated with a first timepoint and (2) sequencing reads from the individual associated with a second timepoint; and (b) administering a cancer type-specific test if the rate of methylation status change is greater than a reference rate of methylation status change.

In some embodiments, provided are methods of quantifying identifiers of cancer risk in an individual, comprising: (a) generating a rate of mutation accumulation using (1) sequencing reads from the individual associated with a first timepoint and (2) sequencing reads from the individual associated with a second timepoint (b) generating a rate of methylation status change using (1) sequencing reads from the individual associated with the first timepoint and (2) sequencing reads from the individual associated with the second timepoint; and (c) administering a cancer type-specific test if the rate of mutation accumulation is greater than a reference rate of mutation accumulation and/or if the rate of methylation status change is greater than a reference rate of methylation status change.

In certain embodiments, the sequencing reads comprise sequence data for non-cancer risk-associated genes. In certain embodiments, the sequencing reads comprise sequence data for specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing reads comprise sequence data for intronic regions. In certain embodiments, the rate of mutation accumulation comprises rate data for a total number of mutations as a function of time, or wherein the rate of methylation status change comprises rate data for a total number of methylation status changes as a function of time.

In some embodiments, provided are methods of processing polynucleotides from the genome of an individual useful for quantifying mutation accumulation in the genome, comprising: (a) sequencing the polynucleotides from the genome of the individual at a first timepoint, and generating sequencing reads associated with the first timepoint; (b) sequencing the polynucleotides from the genome of the individual at a second timepoint, and generating sequencing reads associated with the second timepoint; and (c) quantifying the mutation accumulation in the genome at the first timepoint and the second timepoint and generating a rate of mutation accumulation using (1) the sequencing reads associated with the second timepoint and (2) the sequencing reads associated with the first timepoint.

In some embodiments, provided and described herein are methods of processing polynucleotides from the genome of an individual useful for quantifying methylation status change in the genome, comprising: (a) sequencing the polynucleotides from the genome of the individual at a first timepoint, and generating sequencing reads associated with the first timepoint; (b) sequencing the polynucleotides from the genome of the individual at a second timepoint, and generating sequencing reads associated with the second timepoint; and (c) quantifying the methylation status change in the genome at the first timepoint and the second timepoint and generating a rate of methylation status change using (1) the sequencing reads associated with the second timepoint and (2) the sequencing reads associated with the first timepoint.

In some embodiments, provided and described herein are methods of processing polynucleotides from the genome of an individual useful for quantifying mutation accumulation and methylation status change in the genome, comprising: (a) sequencing polynucleotides from the genome of the individual at a first timepoint, and generating sequencing reads associated with the first timepoint; (b) sequencing polynucleotides from the genome of the individual at a second timepoint, and generating sequencing reads associated with the second timepoint; (c) quantifying the mutation accumulation in the genome at the first timepoint and the second timepoint and generating a rate of mutation accumulation using (1) the sequencing reads associated with the second timepoint and (2) the sequencing reads associated with the first timepoint; and (d) quantifying the methylation status change in the genome at the first timepoint and the second timepoint and generating a rate of methylation status change using (1) the sequencing reads associated with the second timepoint and (2) the sequencing reads associated with the first timepoint.

In certain embodiments, the sequencing reads comprise sequence data for non-cancer risk-associated genes. In certain embodiments, the sequencing reads comprise sequence data for specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing reads comprise sequence data for intronic regions. In certain embodiments, the rate of mutation accumulation comprises rate data for a total number of mutations as a function of time, or wherein the rate of methylation status change comprises rate data for a total number of methylation status changes as a function of time. In certain embodiments, the sequencing comprises sequencing non-cancer risk-associated genes. In certain embodiments, the sequencing comprises sequencing specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing comprises sequencing intronic regions. In certain embodiments, the sequencing comprises long read sequencing. In certain embodiments, the polynucleotides comprise genomic DNA or amplification products thereof.

In some embodiments, provided and described herein are methods of detecting cancer, comprising: administering a cancer type-specific test to an individual having an increased quantity or rate of mutation accumulation, wherein the increased quantity or rate of mutation accumulation is or has been determined by: a rate of mutation accumulation using (1) sequencing reads from the genome of the individual associated with a first timepoint and (2) sequencing reads from the genome of the individual associated with a second timepoint, wherein the quantity or rate of mutation accumulation is greater than a reference rate.

In some embodiments, provided are methods of detecting cancer, comprising: administering a cancer type-specific test to an individual having an increased quantity or rate of methylation status change, wherein the increased quantity or rate of methylation status change is or has been determined by: a rate of methylation status change using (1) sequencing reads from the genome of the individual associated with a first timepoint and (2) sequencing reads from the genome of the individual associated with a second timepoint, wherein the quantity or rate of methylation status change is greater than a reference rate.

In some embodiments, provided are methods of detecting cancer, comprising: administering a cancer type-specific test to an individual having an increased quantity or rate of mutation accumulation and methylation status change, wherein the increased quantity or rate of mutation accumulation and methylation status change is or has been determined by: a rate of mutation accumulation using (1) sequencing reads from the genome of the individual associated with a first timepoint and (2) sequencing reads from the genome of the individual associated with a second timepoint, wherein the quantity or rate of mutation accumulation is greater than a reference rate; and a rate of methylation status change using (1) sequencing reads from the genome of the individual associated with a first timepoint and (2) sequencing reads from the genome of the individual associated with a second timepoint, wherein the quantity or rate of methylation status change is greater than a reference rate.

In certain embodiments, the sequencing reads comprise sequence data for non-cancer risk-associated genes. In certain embodiments, the sequencing reads comprise sequence data for specific loci within the genome of the individual. In certain embodiments, the sequencing reads comprise sequence data for cancer-specific loci within the genome of the individual. In certain embodiments, the sequencing reads comprise sequence data for intronic regions In certain embodiments, the rate of mutation accumulation comprises rate data for a total number of mutations as a function of time, or wherein the rate of methylation status change comprises rate data for a total number of methylation status changes as a function of time. In certain embodiments, the cancer-specific test is selected using a mutational signature profile generated using the sequencing reads associated with the second timepoint, or wherein the cancer-specific test is selected using a methylation profile generated using the sequencing reads associated with the second timepoint.

### Sequencing and Sequencing Reads

In some embodiments, the method comprises whole genome sequencing, wherein all or substantially all (e.g., ≥60%) of the genome is sequenced. In some embodiments, the sequencing comprises long read sequencing.

In certain embodiments, the sequencing comprises whole gen. In certain embodiments, the sequencing comprises sequencing specific loci within the genome of the individual. In certain embodiments, the specific loci comprise (i) a region having an increased frequency of mutations, (ii) an increased rate of local mutation accumulation (e.g., compared to other regions of the genome), or (iii). an increased rate of methylation status change. In certain embodiments, the sequencing comprises sequencing intronic regions. In certain embodiments, the sequencing comprises long read sequencing. In certain embodiments, the polynucleotides comprise genomic DNA or amplification products thereof.

In certain embodiments, the sequencing reads comprise sequence data for specific loci within the genome of the individual. In certain embodiments, the specific loci comprise (i) a region having an increased frequency of mutations, (ii) an increased rate of local mutation accumulation (e.g., compared to other regions of the genome), or (iii) an increased rate of methylation status change.

In some embodiments, sequencing is performed monthly to yearly. In certain embodiments, sequencing is performed once a month. In certain embodiments, sequencing is performed every three months. In certain embodiments, sequencing is performed once every six months. In certain embodiments, sequencing is performed once a year. In certain embodiments, sequencing is performed twice a year. In certain embodiments, sequencing is performed three time a year. In certain embodiments, sequencing is performed four times a year. In certain embodiments, sequencing is performed six times year.

In some embodiments, sequencing refers to and encompasses a method by which the identity of at least 10 consecutive nucleobases (e.g., at least 50, at least 100, at least 500, at least 1,000, at least 10,000, at least 100,000, at least 200,000, or at least 300,000 or more consecutive nucleobases) of a polynucleotide is obtained. In certain embodiments, sequencing comprises next-generation sequencing (NGS) or high-throughput sequencing, which generally refer to and encompass, but not limited to, massively parallel signature sequencing, high throughput sequencing, sequencing by ligation (e.g., SOLiD sequencing), proton ion semiconductor sequencing, DNA nanoball sequencing, single molecule sequencing, and nanopore sequencing parallelized sequencing-by-synthesis or sequencing-by-ligation platform nanopore sequencing methods, or electronic-detection based methods, or single molecule fluorescence-based methods. Further examples of sequencing techniques include whole-genome sequencing targeted sequencing, single molecule real-time sequencing, electron microscopy-based sequencing, transistor-mediated sequencing, direct sequencing, random shotgun sequencing, Sanger dideoxy termination sequencing, sequencing by reversible dye terminator, paired-end sequencing, near-term sequencing, exonuclease sequencing, sequencing by ligation, short-read sequencing, single-molecule sequencing, sequencing-by-synthesis, real time sequencing, reverse-terminator sequencing, ion semiconductor sequencing, nanoball sequencing, nanopore sequencing, 454 sequencing, Solexa Genome Analyzer sequencing, miSeq (Illumina), HiSeq 2000 (Illumina), HiSeq 2500 (Illumina), Illumina Genome Analyzer (Illumina), Ion Torrent PGM™ (Life Technologies), MinlON™ (Oxford Nanopore Technologies), GridON™, PromethION™, real-time SMRT™ technology (Pacific Biosciences), the Probe-Anchor Ligation (cPAL™) (Complete Genomics/BGI), SOLiD® sequencing, MS-PET sequencing, mass spectrometry, and a combination thereof. In certain embodiments, sequencing comprises detecting the sequencing product using an instrument, for example, an ABI PRISM® 377 DNA Sequencer, an ABI PRISM® 310, 3100, 3100-Avant, 3730, or 3730x1 Genetic Analyzer, an ABI PRISM® 3700 DNA Analyzer, or an Applied Biosystems SOLiD™ System (all from Applied Biosystems), or a Genome Sequencer 20 System (Roche Applied Science).

In some embodiments, sequencing reads generally refer to and encompass nucleobase sequences (sequence data) produced by a sequencing reaction and/or a sequence obtained from a portion of a nucleic acid sample. In certain embodiments, sequencing reads comprise nucleobase sequences having a length of 10 or greater, 50 or greater, 250 or greater, 500 or greater, 1,000 or greater, 5,000 or greater, 10,000 or greater, 50,000 or greater, 100,000 or greater, 200,000 or greater, or 300,000 or greater. In certain embodiments, the sequence reads comprise long read sequence data (e.g., average reads lengths greater than 500 nucleobases).

### Quantity and Rate of Mutation Accumulation

In some embodiments, the methods comprise generating the quantity of mutation accumulation (e.g., relative to reference sequence reads). In some embodiments, the methods comprise generating the rate of mutation accumulation (e.g., relative to reference sequence reads) as a function of time.

In some embodiments, generating the quantity or rate of mutation accumulation further comprises generating a mutation signature profile. For example, in certain embodiments, the mutation signature profile characterizes the type of mutation and probable source (e.g., see FIG. 16). In certain embodiments, generating the quantity or rate of mutation accumulation further comprises quantifying the rate of mutation accumulation for specific mutational signatures within the mutation signature profile.

In some embodiments, mutations generally refer to and encompass one or more nucleobase/tide variants and/or indels. In certain embodiments, a mutation comprises a single nucleotide variant, an indel, or both. In certain embodiments, single nucleotide variants (SNVs) generally refer to and encompass a substitution of one nucleotide to a different nucleotide at a position of a nucleotide sequence (e.g., relative to a control or reference sequence). For example, a substitution from a first nucleobase X to a second nucleobase Y represents a substitution and can be denoted as “X>Y.” By way of further example, a cytosine to thymine SNV can be denoted as “C>T.” In certain embodiments, indels generally refer to and encompass an insertion or deletion of one or more nucleobases in the sequence a nucleic acid molecule (e.g., relative to a control or reference sequence).

In some embodiments, nucleic acids or polynucleotides generally refer to and encompass a covalently linked sequence of nucleotides (e.g., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules, or amplification products thereof. In certain embodiments, nucleic acids or polynucleotides generally refer to and encompass, without limitation, single- and double-stranded polynucleotide.

In some embodiments, a reference genome or reference sequence or control sequence refers to any particular known genome sequence, whether partial or complete, that can reference identified sequences from an individual. For example, a reference genome can a prior determined genome of an individual. By way of further example, a reference genome can be a known reference genome, e.g., as found at the National Center for Biotechnology Information at ncbi.nlm.nih.gov. In certain me embodiments, a reference rate comprises a prior measured rate. In certain me embodiments, a reference rate comprises a threshold value (e.g., value greater than a baseline rate).

In certain instances, it should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus. In additional instances, the problem is compounded because reliable aneuploidy calls generally require mapping thousands (e.g., at least about 10,000) or even millions of reads to one or more chromosomes.

### Quantity and Rate of Methylation Status Change

In some embodiments, the methods comprise generating the quantity of methylation status change (e.g., relative to reference sequence reads). In some embodiments, the methods comprise generating the rate of mutation accumulation (e.g., relative to reference sequence reads) as a function of time. In some embodiments, both the rate of mutation accumulation and the rate of methylation status change are utilized in the methods described herein.

In some embodiments, generating the quantity or rate of methylation status change further comprises generating a methylation profile. For example, in certain embodiments, the mutation signature profile characterizes loci of methylation status change.

In some embodiments, DNA methylation refers to and encompasses an epigenetic mechanism characterized by the addition of a methyl group to cytosine nucleobases within genomic DNA (e.g., typically within CpG islands). In certain instances, DNA methylation modifies the function of the genes and affects gene expression. In certain embodiments, DNA methylation comprises the covalent addition of the methyl group at the 5-carbon of the cytosine ring resulting in 5-methyl cytosine (5-mC). In certain embodiments, the methyl group can be further modified to hydroxymethyl cytosine (5-hmC) by the addition of a single hydroxyl moiety. In some embodiments, a methylated cytosine or MeC generally refers to and encompasses to 5-mC, 5-hmC, or both.

In some embodiments, determining methylation status or a methylation level of DNA comprises the methylation status-dependent conversion of to distinguish between methylated and non-methylated CpG dinucleotide sequences. In certain embodiments, methylation of CpG dinucleotide sequences can be measured by employing cytosine conversion-based technologies, which rely on methylation status-dependent chemical modification of CpG sequences within isolated genomic DNA, or fragments thereof, followed by DNA sequence analysis. Exemplary chemical reagents that are able to distinguish between methylated and non-methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified. Bisulfite-treated DNA can subsequently be analyzed by sequencing.

In some embodiments, a reference genome or reference sequence or control sequence refers to any particular known genome sequence, whether partial or complete, that can reference identified sequences from an individual. For example, a reference genome can a prior determined genome of an individual. By way of further example, a reference genome can be a known reference genome, e.g., as found at the National Center for Biotechnology Information at ncbi.nlm.nih.gov. In certain me embodiments, a reference rate comprises a prior measured rate. In certain me embodiments, a reference rate comprises a threshold value (e.g., value greater than a baseline rate).

In certain instances, it should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus. In additional instances, the problem is compounded because reliable aneuploidy calls generally require mapping thousands (e.g., at least about 10,000) or even millions of reads to one or more chromosomes.

### Cancer Screening and/or Prevention

In some embodiments, a generalized cancer screening refers to and encompasses screening or diagnostic procedures used to identify cancer. In certain embodiments, include imaging, laboratory tests (e.g., including tests for tumor markers), tumor biopsy, endoscopic examination, surgery, or genetic testing. In certain embodiments, the laboratory tests comprise blood tests, complete blood counts, urinalysis, or the detection of tumor markers. In certain embodiments, imaging includes diagnostic imagining techniques, and comprises X-ray, computed tomography scan (e.g., a CT scan or computed axial tomography or CAT scan), bone scan, lymphangiogram (LAG), mammogram, reflection imagine (e.g., ultrasound), or emission imaging (e.g., magnetic resonance imaging). In certain embodiments, endoscopic examination comprises cystoscopy (also called cystourethroscopy), colonoscopy, endoscopic retrograde cholangiopancreatography (ERCP), esophago-gastroduodenoscopy (also called EGD or upper endoscopy), or sigmoidoscopy. In certain embodiments, surgical testing comprises a biopsy, e.g., endoscopic biopsy, bone marrow biopsy, excisional or incisional biopsy, fine needle aspiration biopsy, punch biopsy, shave biopsy, or skin biopsy. In certain embodiments, the genetic test comprises US FDA submission/number K131508, K123951, K091960, P160040, P170005, P170041, DEN130010, DEN140044, K100015, P150041, K962873, K033982, K013785, K011031, DEN170046, K130010, K101454, K081092, K080252, K070675, K062694, K210973, P100027, P100024, P190031, P060017 S001-S004, P050045 S001-S004, P040005, P040030, P050040, P940004, P980018, P980024, P040005, P190001, P190004, DEN200001, K130313, K010288, K972200, DEN130018, K163367, K953591, K954214, DEN160003, K190076, K181661, K173492, P130017, P110027/P110030, P190026, P130001, P140023, P160038, DEN170030, K182784, P200019, P200014, P200014, DEN190023, K192944, K073482, DEN170070, K200009, K132978, P200011, K200129, P110020, P120014, DEN170080, H140005, DEN160028, K172287, K203748, P110012, P120019, P120022, P150044, P150047, P160045, P140020, P160018, DEN180028, DEN180028, K193492, K221885, DEN130011, DEN130042, K130775, K101185, P100033, K211499, K120489, K092967, K080896, K173839, DEN170058, P170019, P210011, P200006, P190014, K202304, K190661, K192063, P190032, P200010, P180043, DEN190035, or DEN210011.

In some embodiments, cancer or a tumor generally refers to and encompasses the physiological condition in mammals that is typically characterized by unregulated cell growth. In certain embodiments, a cancer comprises any malignant and/or invasive growth or tumor caused by abnormal cell growth. In certain embodiments, the cancer comprises a solid tumors (e.g., generally named for the type of cells that form the tumor). In some embodiments, the cancer comprises a liquid tumor (e.g., cancer of blood, bone marrow, or the lymphatic system).

### Individuals

In some embodiments, individual is synonymous with patient and/or subject and includes and/or refers to a human. However, examples are not limited to humans and include, chimpanzees, marmosets, cows, horses, sheep, goats, pigs, rabbits, dogs, cats, rats, mice, guinea pigs, and the like. The individual is typically a human and can be a human that has been diagnosed as needing to treat a disease or condition as disclosed herein.

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

### EXAMPLES

### Example I—Serial Testing to Determine Variability in Molecular Alterations Using Long Read Sequencing

### Overview

Cancer initiation occurs when a cell acquires and accumulates certain mutations in genes involved in the regulation of cell processes. Under normal circumstances, the body is primed to prevent those multiple mutations from leading to cancer. However, when the body is under acute or chronic stress, that safeguarding process may become less dependable. To begin understanding the processes that control the accumulation of somatic mutation over a person's lifetime, whole genome sequencing of DNA obtained from whole blood from prospectively enrolled healthy individuals with and without a previous history of cancer and cancer patients was performed. Each individual's DNA was sequenced using Oxford Nanopore Technologies' Long Read Sequencing (LRS) technology multiple times at regular intervals (3 months or 6 months, depending on cohort assignment) to measure changes in the overall number single nucleotide variants (sSNVs) and to further characterize them. The amount and types of sSNVs at each timepoint were calculated which allowed for the identification of patterns of change along the chronological age of the participants (years) as well as within the shorter time frame of our sampling scheme (months). This study showcases the power of LRS for broad, longitudinal sampling of multiple molecular markers and its suitability for detecting coarse-grained differences with relevance to health.

### Introduction

Throughout human life, healthy cells accumulate somatic mutations due to intrinsic and extrinsic forces, a process that varies between both individuals and tissues. Studies have repeatedly shown that mutations involved in the regulation of cell processes and associated with the development of cancer as precursors, sometimes years before the appearance of the disease. A well-known example are mutations present in both healthy skin and cancerous lesions, sharing a mutational signature associated with UV damage which are shown to accumulate with age. The human body is exquisitely primed to prevent those multiple mutations from leading to cancer through critical cellular regulation routes. However, when the body is under acute or chronic stress, those safeguarding process may become less dependable and the affected tissues may be more prone to develop cancer.

Decades of research have revealed mechanisms of DNA repair and replication in mutagenesis, yet surprisingly little is known about the rate of accumulation of somatic mutations caused by normal cellular processes over an individual's lifetime and its fluctuations in response to intrinsic or extrinsic factors. Clonal hematopoiesis (CH), can occur when mutations caused by environmental triggers (e.g. smoking or cytotoxic therapies), inability to rectify DNA replication errors, or a combination of both extrinsic stress and intrinsic repair rate, result in detectable clonally expanded populations of hematopoietic stem cells. While CH can result in hematologic malignancy, cooperative mutations in additional genes are required to induce malignant transformation. Population-scale data have revealed that on average, ~1.3 somatic exonic mutations are acquired per hematopoietic stem cell per decade. In addition, as hematopoietic cells travel to different tissues of the body, affecting homeostasis among a number of different organ systems cells, recent studies have demonstrated the potential for these altered immune cells to impact risk for cardiovascular disease, diabetes, cancer, and other inflammatory syndromes. Though CH was found to be associated with aging, studies investigating CH and its clinical significance captures only a snapshot of the somatic genome landscape in time, making it hard to evaluate the dynamics of somatic genomes and its effect on human health. Therefore, investigating the dynamism of the somatic landscape and its association with clinical manifestations, as it may unfold a new paradigm for evaluating general health and in particular the body's ability to quench mutations.

Initial efforts sequencing of peripheral blood DNA in a cohort of 12,380 Swedish patients was among the first to identify a large fraction of CH carriers that did not have known disease driving mutations with similar results were found in ~11,262 Icelandic patients using short-read sequencing (XXX, XXX). These studies emphasize the need to better understand the process of accumulation of somatic mutations and their effect on general health.

The classical unidimensional paradigm of genotype-phenotype association has undergone significant changes in the last few years with the introduction of advanced molecular profiling platforms that enabled researchers to start addressing multi-dimensional molecular associations between somatic mutation rates and the contribution of endogenous and exogenous mutational processes across normal tissues. For example, a study using long-read sequencing (LRS) of whole blood and heart tissue from ~3,622 patients provided an understanding of the effects of sequence variation on disease phenotype, particularly genomic regions thus far inaccessible using short-read sequencing alone.

These studies, however, fail to assess the dynamic aspect of mutation accumulation and whether its rate is responsive to environmental and physiological changes. The paucity of serial longitudinal samples have limited study designs and methodologies used to establish these associations as genome sequences and cancer risk vary between ancestry groups. As studies detail new genotypic and phenotypic associations, researchers and clinicians are confronted with the question of what preventative and therapeutic interventions could be taken to mitigate disease risk.

In the clinical setting, blood-derived markers, mainly proteins and DNA, are used to identify and evaluate systemic conditions and/or pathologies and to monitor response to treatment for those conditions. Molecular approaches looking at blood, mainly characterizing molecular features of peripheral immune cells to detect pathological process as cancer, have been demonstrated in several studies showing the potential of utilizing molecular data from peripheral blood cells to capture systemic conditions. This pilot study is driven by the hypothesis that an individual's rate and accumulation of mutations, in particular somatic single nucleotide variants (sSNVs), can be measured over time using LRS from whole blood.

### Study Design: Participants and Participant Data

This study was a non-interventional prospective pilot study recruiting participants for the collection of blood samples over time for whole genome sequencing.

A total of 103 male and female adults' participants were enrolled. Patients treated at the outpatient clinic at the Ellison Institute of Technology (EIT) were approached and offered to participate in the study. In addition, EIT staff members were also offered the chance to voluntarily participate in the study. The study cohort includes active cancer patients defined as the presence of local or advanced disease in the last 6 month (regardless of treatment status), patients with a history of cancer that were definitely treated and are disease free for at least 6 months, and individuals without a history of cancer. Individuals without symptoms of active infection at enrollment were allowed to participate. The study was approved by Western Institutional Review Board (WIRB)—Copernicus Group (WCG).

In accordance with the approved protocol, participants' demographics were recorded at the time of the first draw. This information was anecdotal information only if the participant is not a patient. For the clinic patients' clinical data was also gathered if available from medical record review including clinical/pathologic staging (if applicable), results of imaging studies (if applicable), results of diagnostic blood tests, treatment history, current treatments, current medications, and participant demographics. For the non-patients composed of the EIT staff volunteers taking part in the study, clinical data was collected using designated questionnaires capturing medical conditions including current and past medical history, allergies, medications, herbal or vitamin supplements, family history of cancer and/or chronic diseases.

### Sample Processing

Informed Consent covers the collection of up to 10 ml of blood for this study at each visit. For patients, efforts were made to obtain research blood samples immediately following venipuncture or vascular access performed as part of standard-of-care procedures. Blood was collected into K2EDTA tubes, put in ice and then aliquoted and frozen in −80° C. Participant had multiple specimens collected over the duration of this study. The interval of blood draws varies between the study participants, as active cancer patients, could receive a study draw (up to 10 ml) every scheduled visit, optimally at least 2 draws during cytotoxic therapy, whereas participants not receiving treatment (individuals with a history of cancer being followed at the clinic and participant without any history of cancer) may receive a study draw (up to 10 ml) every 6 to 12 months. Specimens collected at each visit were labeled with an Ellison Biospecimen ID and linked to a deidentified Ellison ID according to the study protocol.

DNA samples sequenced in this study were isolated from whole blood. DNA from whole blood was extracted using either the MagAttract HMW DNA Kit (Qiagen) or the Quick-DNA MagBead Plus Kit (Zymo) following the manufacturer's protocol (see supplementary table SX for sample processing details). Isolated DNA samples' concentrations were quantified using a Qubit 2.0 Fluorometer (Life Technologies) and purity was assessed using NanoDrop One (Thermo Scientific). Lymphoblastoid cell line purified DNA for the reference genome HG002 was purchased from Coriell (ID NA24385).

After extraction, DNA was fragmented using FastPrep-96 Fragmentation and Agilent 4150 TapeStation. Sequencing libraries were generated using the SQK-LSK110 ligation kit from Oxford Nanopore Technologies (ONT). Sample input DNA was determined based on QC analysis (described below) to ensure consistency and quality of sequencing. For sequencing, samples were loaded onto PromethION R9.4.1 flow cells following ONT's standard operating procedures. Sequencing was performed on the PromethION device for 55 hours and data acquisition time was recorded. Reference genome HG002 was sequenced every time in together with the sequencing of clinical samples.

Sequencing and data acquisition were performed using ONT's MinKNOW v. 22.08.06, 22.10.7 and 23.07.12. Raw signal values were processed in real time using guppy (v. 6.2.7, 6.3.9 and 7.1.4) with a high accuracy model and mapped to the reference chromosomes of the human genome v GRCh38.p13. Adaptive sequencing functionality was used to exclude gene regions harboring pathogenic and likely pathogenic variants according to ClinGen or in the ACMG list of genes with reportable secondary findings (ACMG SF v3.1) including 10,000 bp upstream and downstream of the gene start.

### Somatic Variant Calling

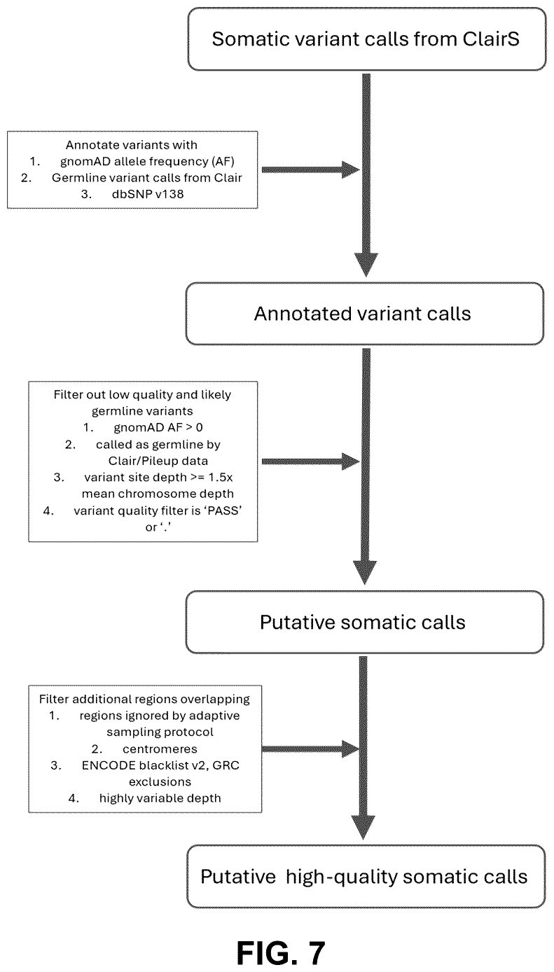

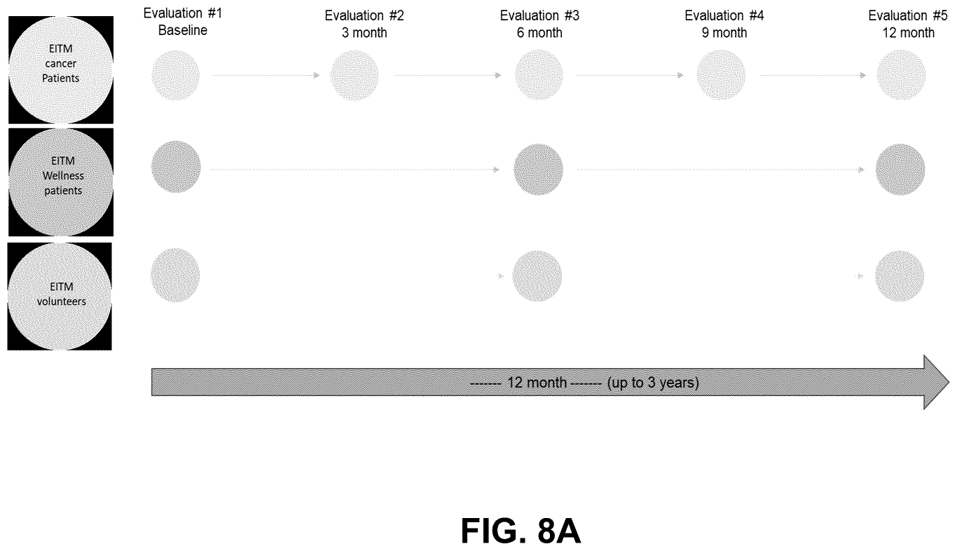

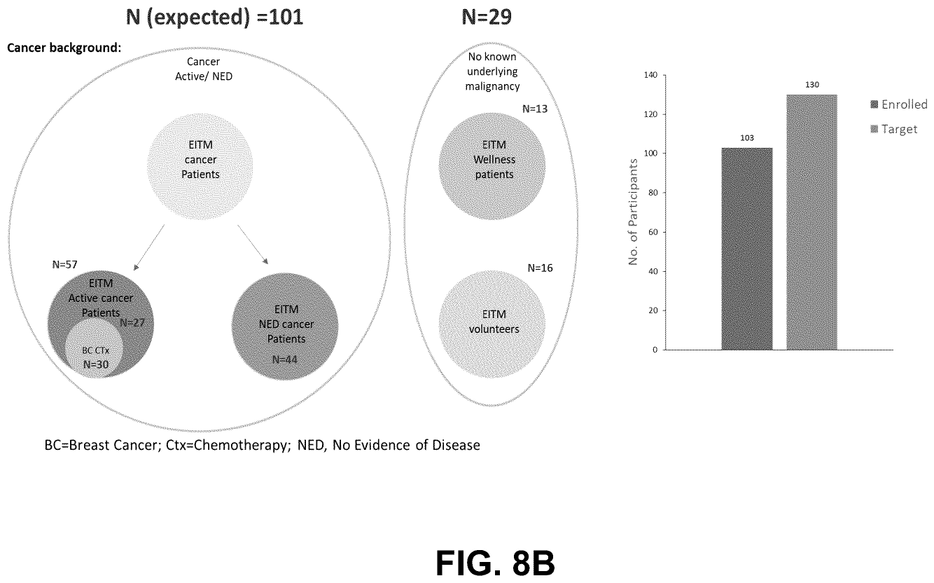

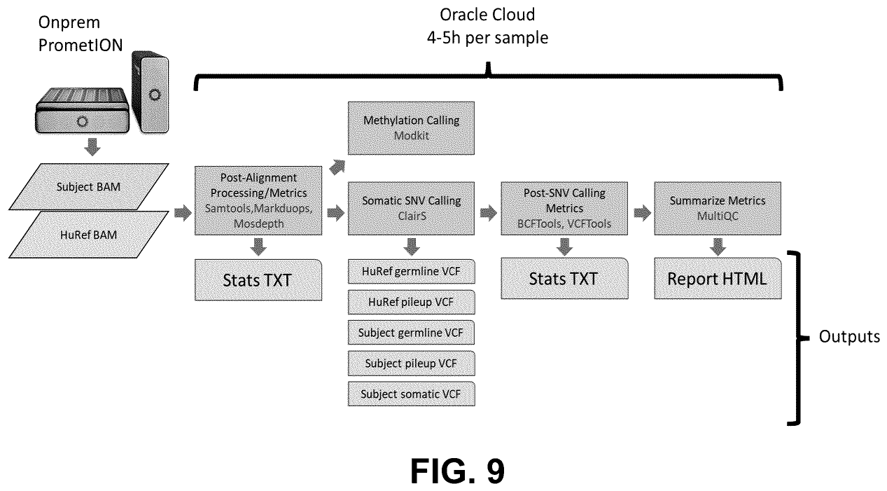

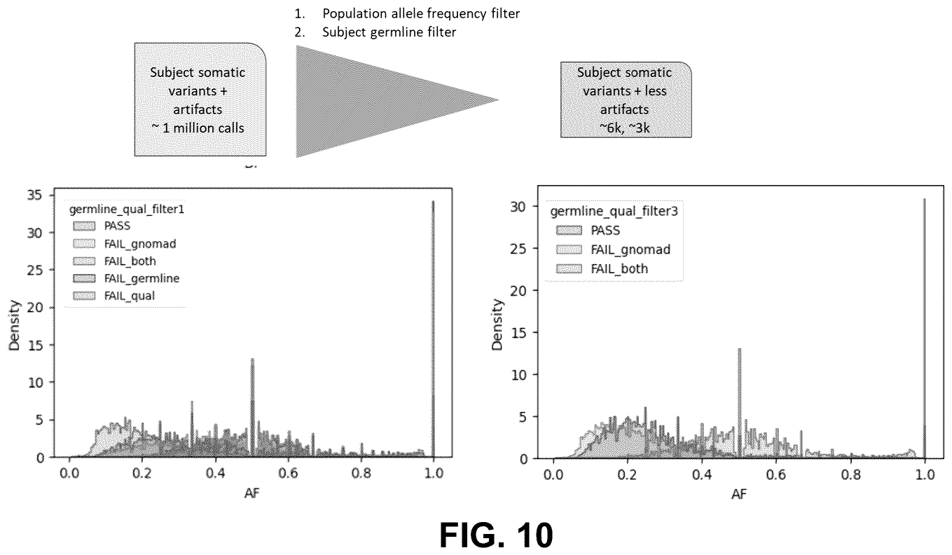

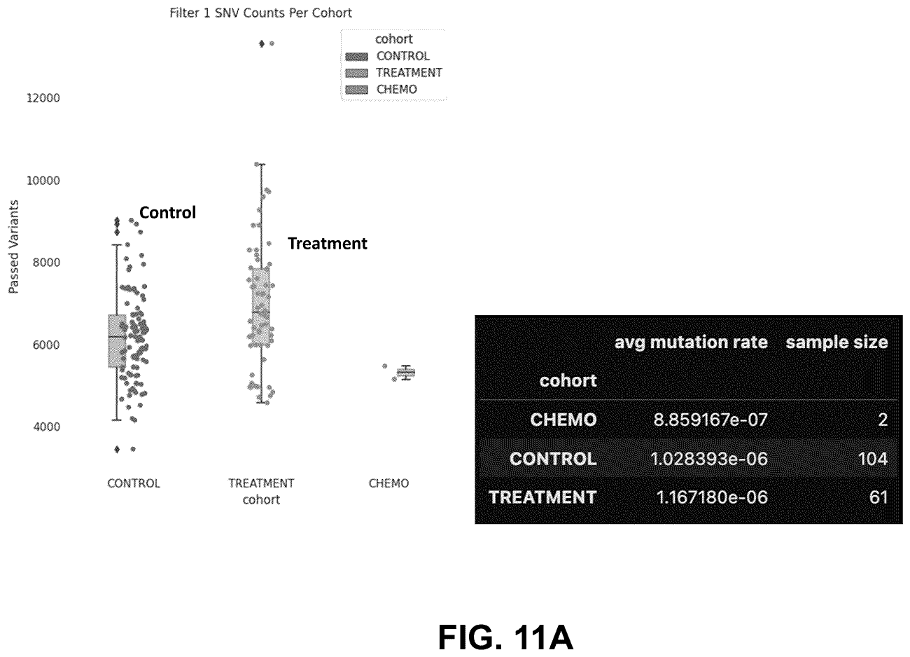

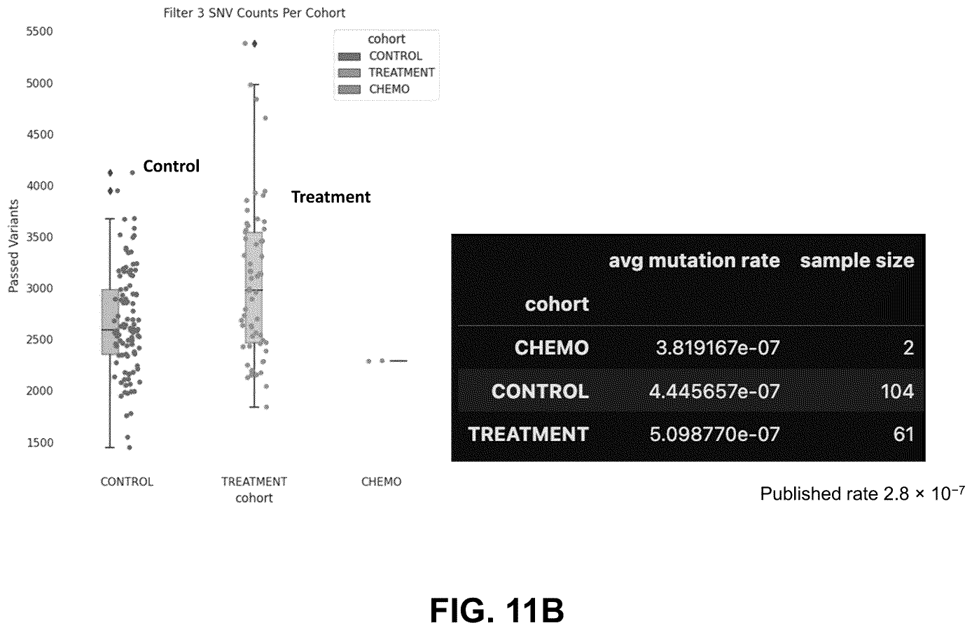

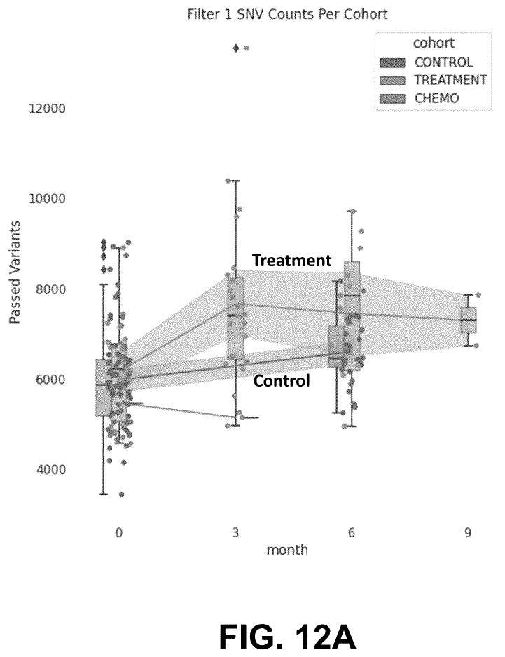

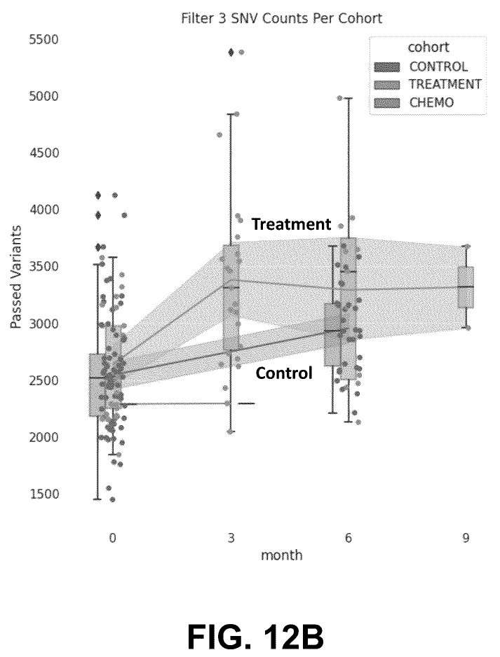

A custom-developed pipeline was developed and implemented using the Nextflow workflow engine. In order to enrich for putative SNVs, ClairS, a deep-learning based somatic variant caller for Long Read sequencing was used followed by multiple filtering steps to remove artifacts and likely germline mutations (Supplementary FIG. 7). In order to identify hard to sequence areas and capture potential batch effects, a well characterize DNA reference genome sample (HG002) was processed in parallel and sequenced alongside each batch of subject samples. The same ClairS pipeline was used on the reference genome to identify all variants and after removing the known germline variants, used to identify sequencing artifacts and errors. The artifact identified in this step were then removed from the list of putative sample SNVs. Next, variant sites with base quality scores lower than 15 are removed as well as any SNV with frequencies above 40% (likely subject-specific germline variants). Finally, putative somatic variants were compared to a list of known SNPs present across human populations (gnomAD v 3.1.2, Chen 2024) any matching sites are removed.

Additionally, a set of locations-based filters were applied to eliminate putative SNVs occurred in sequence regions known to be challenging to sequence (Centromeres: Miga 2014; Repetitive regions: Jurka 2000; ENCODE Blacklist: Amemiya 2019) as well as regions that were empirically determined to have excessive noise level as determined a running window of mean coverage. SNVs that had a depth of coverage greater than 1.5 times the mean depth of coverage for that chromosome were excluded from the analysis. All variants in gene regions associated with reportable findings were removed to minimize the risk of revealing relevant medical information.

These filtering steps reduced the average number of putative SNV per subject from 1.2 million to approximately 50,000 high-confidence sSNVs per subject.

TensorSignatures, a multi-dimensional tensor factorization framework was used for characterizing high-confidence sSNVs in terms of the underlying mutational signatures and associated processes. Additionally, mutational signature associated with sequencing errors and artifacts were generated from the false positive SNVs obtained from the reference genome sequencing. To this aim, TensorSignatures refit with the predefined Pan-Cancer Analysis of Whole Genomes (PCAWG) signatures were applied to the HG002 false positive catalog, which results in a set of falsely discovered signatures and the corresponding exposure matrix. The falsely discovered exposure matrix were analyzed to calculate false discovery rates for each of the 20 PCAWG signatures. From the false discovery rates, an error model that adjusts an arbitrary PCAWG exposure matrix based on the false discovery rates into a corrected exposure matrix was built.

To characterize subject samples, TensorSignatures refit with the PCAWG signatures were applied to each subject's high-confidence sSNV catalogue and applied the error model to adjust the output and calculate a false discovery rate (FDR)-adjusted exposure matrix. The FDR-adjusted exposure matrix was used to model and track subject mutational signatures over time.

### Outcomes: Long Read Sequencing can be Used to Identify and Characterize Low Frequency Single Nucleotide Variants in a Contrived Sample

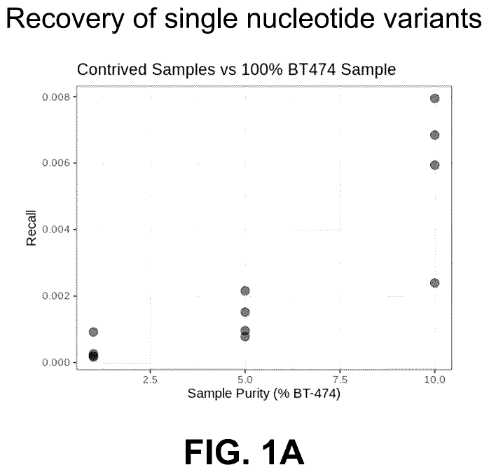

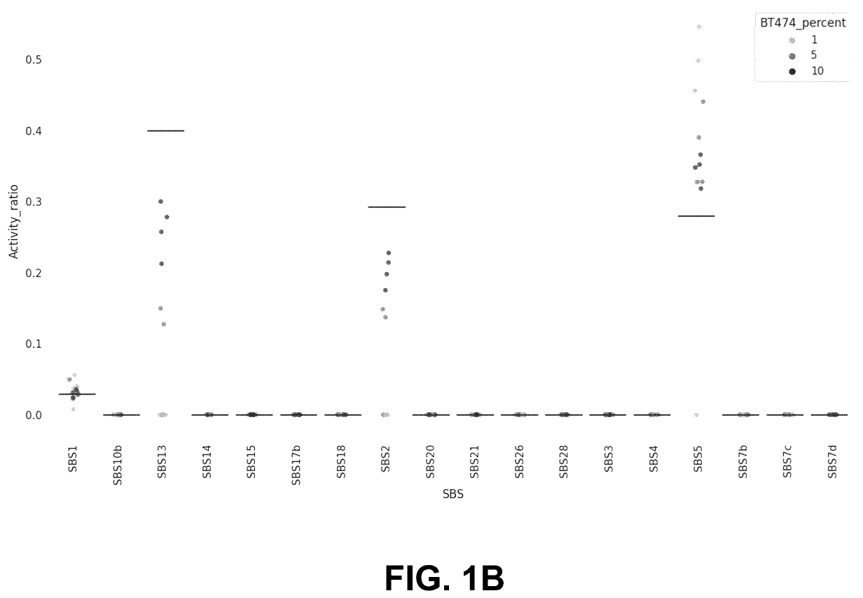

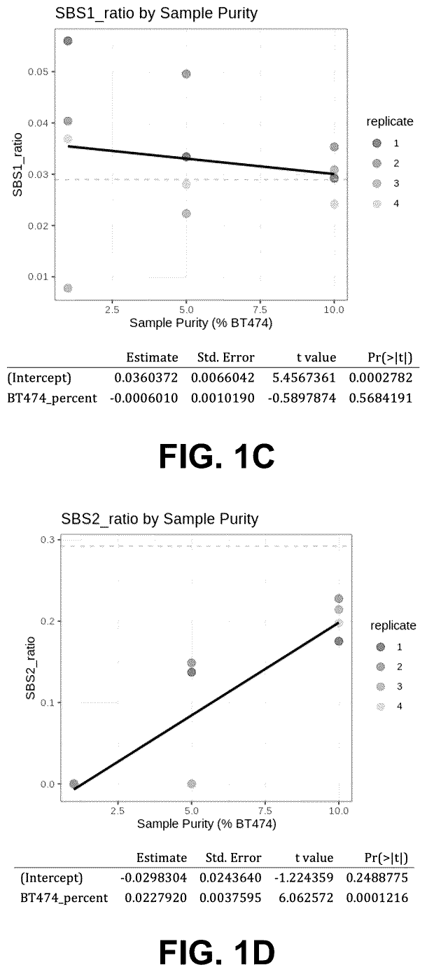

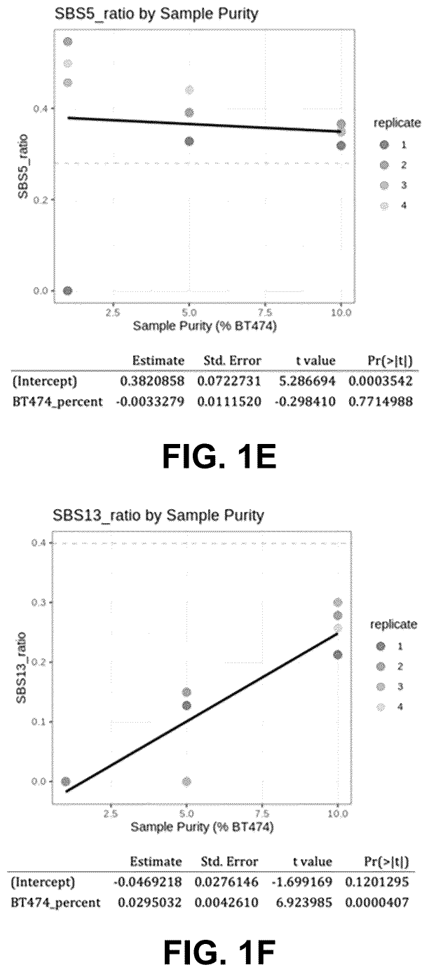

To determine whether LRS-based approaches can capture and characterize low-frequency single nucleotide variants, a contrived sample experiment in which increasing amounts of DNA from a well characterized cancer cell line (BT-474) spiked into a known genomic DNA background (HG001) was tested. BT-474 is a breast cancer-derived cell line characterized by APOBEC activity and displaying an enrichment of single nucleotide variants matching the mutational signatures SBS2 and SBS13. At 10% BT-474 DNA concentration, our pipeline was able to recover an average of 416 (SEM 86) SNVs, corresponding to 0.577% of the number observed in a pure sample of BT-474 DNA sequenced on the same instrument (SNVs common to two replicates). At 5% and 1% BT-474 concentration, the proportions recovered are 0.135% and 0.038% respectively, indicating a roughly linear recovery between 5% and 10% and a drop below accurate quantification below 5% (FIG. 1A).

Moreover, at all frequencies, including at 1%, our signature analysis shows significant enrichment of signatures SBS2 and SBS13 whereas all other signatures remain undetected or at much lower level with the exception of SBS1 and SBS5, which are ubiquitous, clock-like mutational signature. While SBS1 and SBS5 were also observed in BT-474, unlike SBS2 and SBS13, the relative proportions of these two signatures do not change depending on the amount of BT-474 spiked in, suggesting that they might also reflect noise (FIG. 1B-F).

### Outcomes: Aggressive Filtering Results in High-Confidence Somatic SNVs

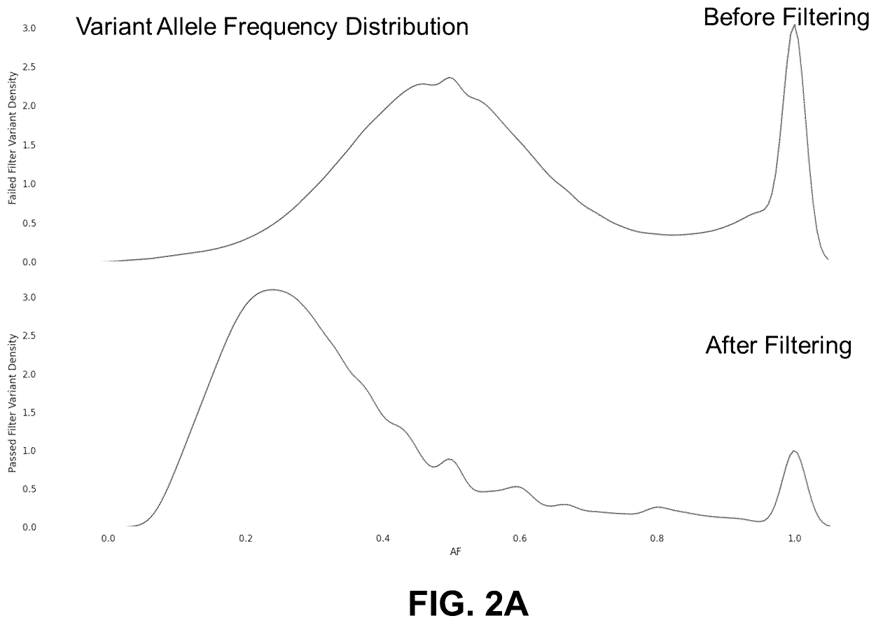

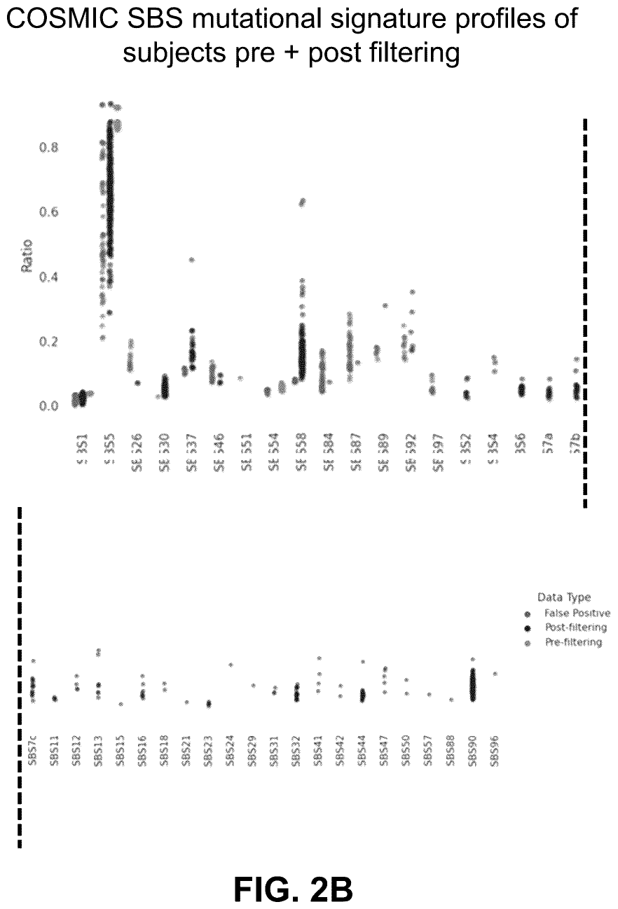

Given the challenge present in identifying and distinguishing somatic variants from germline variants and sequencing artifacts, a set of filtering steps was applied to the initial set of SNV to progressively enrich for somatic SNVs. In total, our characterization pipeline identified 425,459,468 SNVs across 103 subjects (mean SNVs per subject 1,477,289, SD 311,645). These were expected to be composed of bona-fide somatic variants as well as germline variants and sequencing errors and artifacts. The aggregate allele frequency spectrum (FIG. 2A—top) is compatible with this expectation with peaks and shoulders around 0% and 100% frequencies (sequencing errors), a broad peak around 50% (heterozygous germline variants affected by sampling noise) and a diffuse peak at low and intermediate frequencies (between ~1% and ~30%) where an enrichment for somatic variants was expected. After applying our filtering, the 211,530 high-confidence SNVs (mean SNVs per subject 801.25, SD 227.15) with an allele frequency distribution is centered around 20% (FIG. 2A—bottom) remained. The filtered out set of SNV displayed an almost uniform distribution of mutational signatures with limited variation across individuals and just three signatures accounting for 100% of all observed SNV (SBS5 94%, SBS1 4% and SBS54 2%, FIG. 2B).

### Outcomes: High-Confidence Somatic SNVs Display Different Mutational Signatures From Sequencing Artifacts

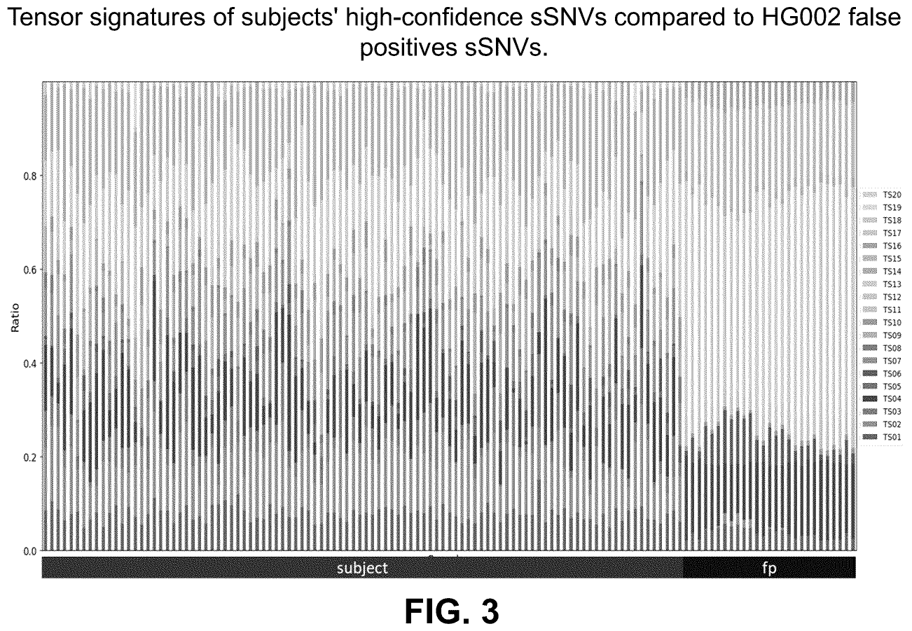

To verify that the high confidence sSNVs are not in fact sequencing artifacts, their mutational signature was compared to the signature derived from false positive SNVs detected in our reference HG002 samples. As HG002 is a very well characterized genome, all (germline) SNV variants are known, which allows classification of all detected SNVs as sequencing errors and artifacts. The reference genome false positive set showed a different profile from all the subject samples (FIG. 3), with significant overrepresentation of certain signatures (e.g. TS06, TS11, TS13, TS15 and TS16) and absence of multiple signatures observed across subject samples (e.g. TS03, TS05, TS07, TS08, TS09, TS11, TS12, TS17, TS18 and TS19). Additionally, the relative proportions of the different signatures are highly variable across subjects and fairly uniform across the different replicate sequencing runs of HG002.

### Outcomes: Interpretable Mutational Signatures Differ Among Individuals

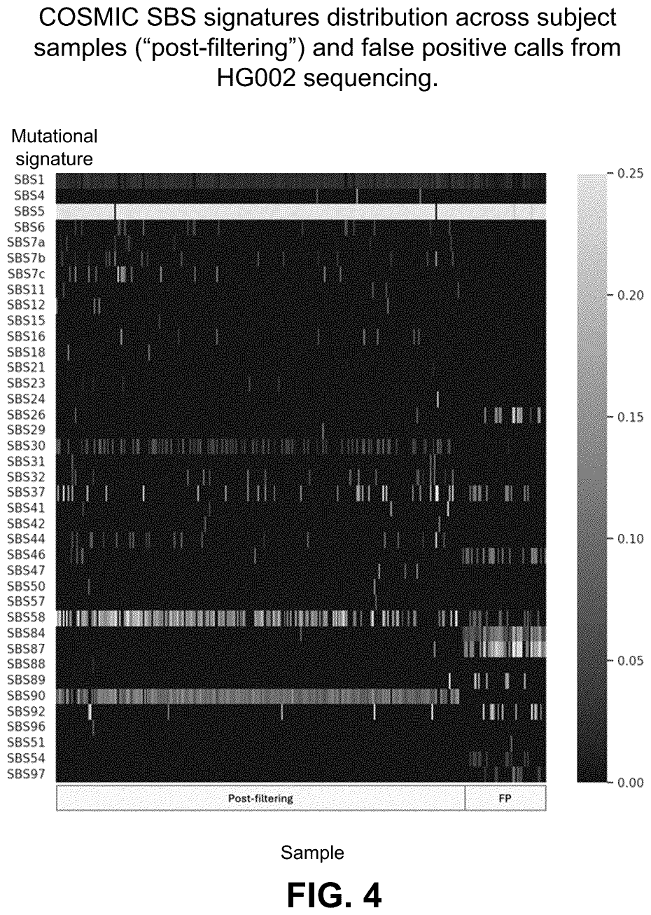

While TensorSignatures are useful for identifying novel patterns, they have limited interpretability and connection to the underlying mutational processes. Conversely, COSMIC's Single Base Substitution (SBSs) mutational signatures have been linked to various mutational processes and exposures. COSMIC SBS signature was used to characterize our samples (FIG. 4).

### Outcomes: The Number of Somatic SNVs Increases Over a Person's Lifetime

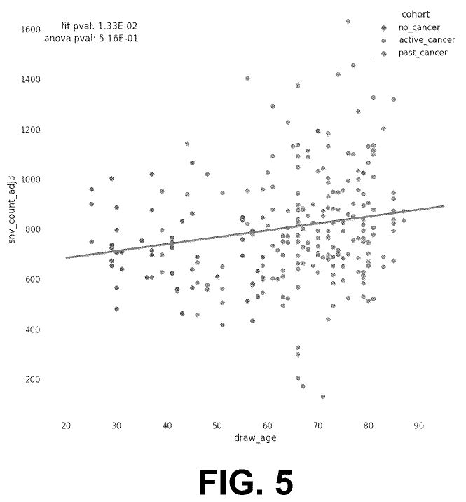

The number of sSNVs is known to increase over a person's lifetime, however, this has primarily been observed by analyzing the RNA of autopsy material or DNA from biopsies of by laborious lab experiment. Recently, an analysis of genomes obtained from the peripheral blood of over 43,000 individuals revealed a collection of sited that are recurrently mutated in a time dependent fashion, meaning that in older individuals more sites are mutated. To address the question of whether a time dependent accumulation of sSNVs can be detected over an individual's lifetime, the corrected number of observed sSNVs was modeled against the individual's age (at first draw). A strong age dependent relationship (p val=0.0027) corresponding to an accumulation of approximately 2.7 sSNVs per year of life (FIG. 5). This rate does not seem to be dependent on the clinical status of the subject, however the cohorts were not designed to conclusively address this question.

### Outcomes: Different Mutational Processes Accumulate at Different Rates

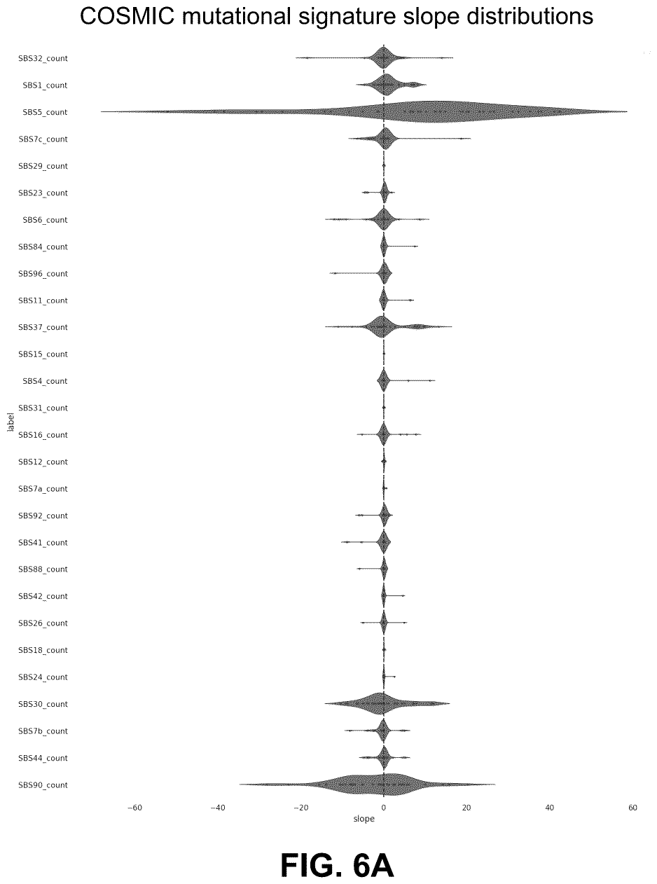

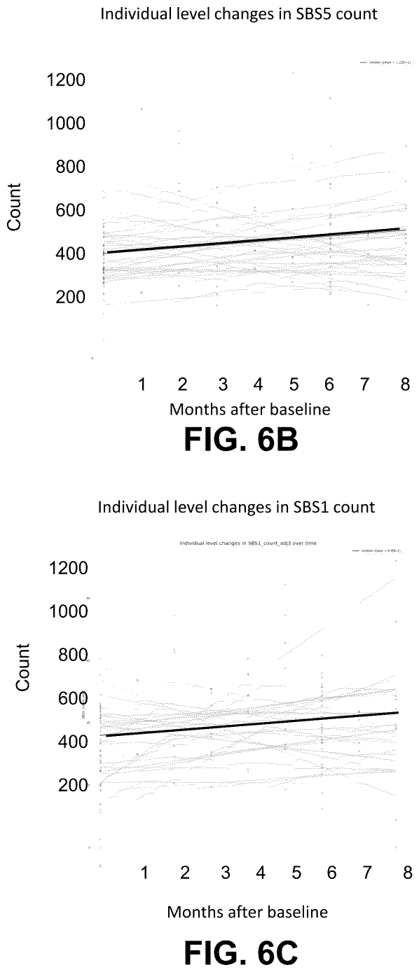

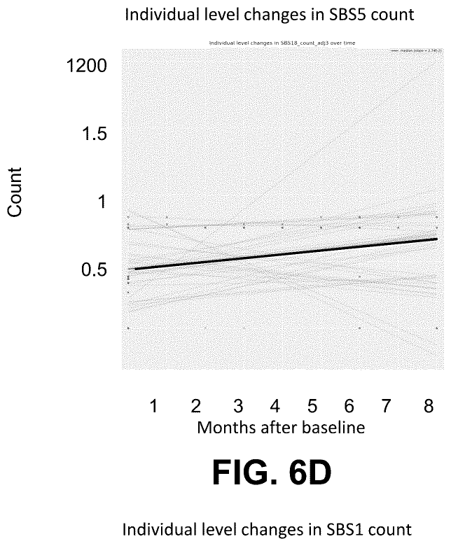

Whether changes in mutation accumulation rate could be observed at shorter time interval was of interest. Given the overall rate of accumulation of sSNVs and the measurement noise, the ability to detect short-time (i.e. within months) changes is limited. Nevertheless, it was reasoned that because the different mutational processes are occurring at different rates, there might be specific signatures that might be better at capturing rapid changes compared to others. For every mutational signature collected, a liner regression was performed on the corrected counts of the signature over time and looked at the distribution of slopes from the linear model. As expected, for most signatures, the distribution of regression slopes across individuals are roughly symmetrical and centered across 0, indicating no temporal accumulation effect. However, for several signatures it was observed that an overall skew towards positive numbers FIG. 6A. In particular, a bias towards positive slopes for SBS5 (FIG. 6B) was observed, a signature known to be associated with age and to be elevated in tobacco users. Similarly, SBS1 (FIG. 6C), a signature associated with the spontaneous deamination of 5-methylcytosine and to be generally correlated with SBS5 also shows a skew towards positive slope values.

### SUMMARY

This Example reports the results of a study aimed at detecting and characterizing somatic single nucleotide variants from whole blood and measure their rate of accumulation of over time.

The accumulation of mutations and the rate of their occurrence are affected by the efficacy of DNA repair mechanisms which are key molecular mechanisms involved in maintaining cellular homeostasis and normal cellular function. The ability to monitor such a fundamental molecular mechanism over time might help us better quantify and monitor an individual's risk for developing cancer and other late-onset diseases. LRS was used to monitor the rate and variability of somatic genomic alteration over time. This experimental approach required special attention and rigorous monitoring of pre-analytical variables to enable reliable individual-level detection of genomic changes across the whole genome. A workflow for serial whole genome LRS from individual's whole blood was developed and established, and was able to quantify and characterize the rate of accumulation of sSNVs across both a long time (lifetime) and short time (months) interval.

Using this approach, changes in specific genomic signatures on a patient level basis and differences in the rate of their accumulation over time were able to be identified. These findings enable the longitudinal monitoring of genomic changes toward the development of “state of the system” personalized cancer prevention programs.

1. A method of quantifying somatic mutations in a genome of an individual, the method comprising:
(a) sequencing polynucleotides from the genome of the individual at a first timepoint and generating:
(i) sequencing reads associated with the first timepoint, and
(ii) a first rate of mutation accumulation using (1) the sequencing reads associated with the first timepoint and (2) a reference sequence; and
(b) sequencing polynucleotides from the genome of the individual at a second timepoint and generating:
(i) sequencing reads associated with the second timepoint, and
(ii) a second rate of mutation accumulation using (1) the sequencing reads associated with the second timepoint and (2) the sequencing reads associated with the first timepoint.

2. The method of claim 1, further comprising, after (b), identifying the individual as having an increased risk of cancer if the second rate of mutation accumulation is greater than the first rate of mutation accumulation.

3. The method of claim 1, wherein:
generating the first rate of mutation accumulation comprises generating the number of mutations present in the sequencing reads associated with the first timepoint relative to the reference sequence; and
generating the second rate of mutation accumulation comprises generating the number of mutations present in the sequencing reads associated with the second timepoint relative to the sequencing reads associated with the first timepoint.

4. The method of claim 1, wherein the genomic mutation accumulation comprises the accumulation of somatic single nucleotide variants (SNVs) present in the sequencing reads.

5. The method of claim 4, where the somatic SNVs are high-confidence somatic SNVs.

6. The method of claim 5, wherein the high-confidence somatic SNVs are obtained by removing SNVs associated with sequencing artifacts detected in control sequencing reads from a control genome, removing putative germline SNVs, removing SNVs having a frequency equal to or greater than about 30%, removing SNVs mapping to known single nucleotide polymorphisms having a population allele frequency greater than a specified threshold (e.g. 0%), or a combination thereof.

7. The method of claim 5, wherein the high-confidence somatic SNVs are obtained by removing SNVs associated with sequencing artifacts detected in control sequencing reads from a control genome, removing putative germline SNVs, removing SNVs having a frequency equal to or greater than about 40%, and removing SNVs mapping to single nucleotide polymorphisms having an allele frequency equal to or greater than about 0.01%.

8. The method of claim 7, wherein substantially all germline SNVs or all germline SNVs are known for the control genome.

9. The method of claim 7, wherein the control genome is sequenced in parallel with (a) and/or (b).

10. The method of claim 1, further comprising:
sequencing polynucleotides from a control genome at the first timepoint and/or the second timepoint, and generating control sequencing reads associated with the first timepoint and/or second timepoint, wherein all or substantially all of the sequence of the control genome is known, including all or substantially all germline SNVs.

11. The method of claim 10, further comprising:
generating a set of variants associated with sequencing artifacts by:
(i) filtering out the known germline SNVs from the control sequencing reads associated with the first timepoint and/or second timepoint; and
(ii) using the sequence of the control genome to identify variants associated with sequencing artifacts in the control sequencing reads associated with the first timepoint and/or second timepoint.

12. The method of claim 11, wherein:
generating the first rate of somatic SNV accumulation and/or the second rate of somatic SNV accumulation comprises:
(i) filtering out putative germline SNVs from the sequencing reads associated with the first timepoint and/or second timepoint;
(ii) removing variants present in the sequencing reads associated with the first timepoint and/or second timepoint that map to the variants associated with sequencing artifacts in the control sequencing reads associated with the first timepoint and/or second timepoint;
(iii) removing SNVs having a frequency equal to or greater than about 40%; and/or
(iv) removing SNVs mapping to known single nucleotide polymorphisms having an allele frequency equal to or greater than about 0.01%.

13. The method of claim 1, wherein sequencing comprises long-read sequencing.

14. The method of claim 1, further comprising: generating a first rate of epigenetic changes associated with the first timepoint and a second rate of epigenetic changes associated with the second timepoint.

15. The method of claim 14, wherein the epigenetic changes comprise changes in genome methylation.

16. The method of claim 1, wherein the sequencing reads comprise sequence data for non-cancer risk-associated genes, cancer risk-associated genes, specific loci and/or genes within the genome of the individual, or a combination thereof.

17. The method of claim 1, wherein generating the rate of genomic mutation accumulation comprises generating comprises rate data for a total number of mutations as a function of time.

18. The method of claim 1, further comprising administering or performing a cancer type-specific test if the rate of mutation accumulation is greater than to a reference rate.

19. A method of processing polynucleotides from the genome of an individual useful for quantifying somatic SNV accumulation, the method comprising:
(a) longitudinally collecting three or more blood and/or plasma samples from the individual at three or more timepoints;
(b) sequencing polynucleotides from the genome of the individual in the three or more blood and/or plasma samples; and
(c) generating a first rate of somatic SNV accumulation and a second rate of mutation accumulation, wherein:
(i) the first rate of somatic mutation accumulation comprises the number of somatic SNV mutations present in sequencing reads associated with a second timepoint relative to a first timepoint, and
(ii) the second rate of somatic mutation accumulation comprises the number of somatic SNV mutations present in sequencing reads associated with a third timepoint relative to the sequencing reads associated with the second timepoint.

20. A method of detecting cancer, the method comprising:
administering a cancer type-specific test to an individual having an increased rate of somatic mutation accumulation, wherein the increased rate of somatic mutation accumulation is or has been determined by:
a second rate of somatic mutation accumulation being greater than a first rate of somatic mutation accumulation, wherein:
the first rate of somatic mutation accumulation comprises the number of somatic mutations present in sequencing reads associated with a first timepoint relative to a reference sequence, and
the second rate of somatic mutation accumulation comprises the number of somatic mutations present in sequencing reads associated with a second timepoint relative to the sequencing reads associated with the first timepoint.
